(19) United States
(12) Patent Application Publication
(10) Pub. No.: US 2005/0071892 A1
(43) Pub. Date: Mar. 31, 2005

(54) Techniques and applications of establishment of SARS-CoV primate model

(76) Inventors: Chuan Qin, Beijing City (CN); Qiang Wei, Beijing City (CN); Hong Jiang, Beijing City (CN); Hua Zhu, Beijing City (CN); Hong Gao, Beijing City (CN)

Correspondence Address: BIRCH STEWART KOLASCH & BIRCH, PO BOX 747, FALLS CHURCH, VA, 22040-0747, US

(21) Appl. No.: 10/875,267

(22) Filed: Jun. 25, 2004

(30) Foreign Application Priority Data
Jun. 27, 2003 (CN) 03148975.3

(51) Int. Cl. A01K 67/027; C12Q 1/70

(52) U.S. Cl. 800/9; 800/14; 435/5

(57) ABSTRACT

This invention relates to a primate animal model for SARS, namely, the techniques and applications of establishment of SARS by using Rhesus monkeys (Macaca Mulata) and Cynomogus Macaques (Macaca fascicularis). The Rhesus monkeys and Cynomogus Macaques free-living in China are infected at least through one route for inoculation: inoculated via nasal cavity, and the dose is 0.5-1 ml 10⁷ TCID₅₀; inoculated via the bronchi, the dose being 0.5-1 ml 10⁶ TCID₅₀; or inoculated intravenously, the dose being 0.5-1 ml 10⁵ TCID₅₀.

### BACKGROUND OF THE INVENTION

1. Field of the Invention

This invention is related to a primate animal model for SARS, namely, the techniques and applications of establishment of SARS by using Rhesus monkeys (Macaca Mulata) and Cynomogus Macaques (Macaca fascicularis).

2. Description of the Related Art

Severe Acute Respiratory Syndrome (SARS) threats badly on human heath. The studies in China and abroad revealed that SARS-CoV pathogen is a mutant from coronavirus. The SARS-CoV is single positive RNA virus with 29742 bp in size, and the frequency of recombinant between RNAs is very high.

We have achieved greatly in studies of pathogen, diagnosis, and resource for SARS in China, however, There are no suitable animal models, e.g. the primate animals available for SARS research. Therefore, the lacking SARS animal models has been a bottleneck for studies on disease mechanism, the selection of preventive or treatment medicines and evaluation on vaccines against SARS-CoV.

There have been primate animal models for different pathogens. However, to infect primate animals with the mutant strain of coronavirus, the SARS pathogen, many techniques remains unknown such as the species of animals, the choice of animal quality, the processing of virus for inoculation, the dosage, the different routes, and virological detection and serological determination after inoculation with SARS-CoV. So far, there was only one short communication published about SARS infection in Cynomogus Macaques (Nature, Vol. 423, 15 May, 2003), the infection was primarily proved, but no more detailed information available on establishment of an animal model for SARS-CoV.

### SUMMARY OF THE INVENTION

The inventors have overcome present technological problems, and have established successfully the SARS infectious animal model with Rhesus monkeys and Cynomogus Macaques free-living in China. The repetition and stability of the models of the primates worked very well, and provide important basic data for research fields of SARS etiology, SARS mechanism, selection of medicine against SRA and vaccine evaluations.

One aspect of this invention is related to a primate animal model for SARS, especially, the establishment of SARS by using Rhesus monkeys and Cynomogus Macaques free-living in China.

All the Rhesus monkeys and cynomogus macaques showed a transient fever after inoculation of SARS-CoV, the SARS coronavirus-specific IgM and IgG was begun to detect positive in the serum of monkeys, virus isolation was positive, and the SARS-CoV RNA was detected positive for 10-15 days using nested RT-PCR in pharyngeal swab samples. The establishment of SARS model set up virological and serological criteria, and served uniquely for SARS related researches including etiology, immunology, pathogenesis, and screening for medicines and vaccines against SARS-CoV, and also presented representative significance in selecting of sensitive animals for SARS-CoV.

Another aspect of this invention is related to the technique for establishment of primate animal model for SARS, especially, the establishment of SARS by using Rhesus monkeys and Cynomogus Macaques free-living in China.

In the practical scheme of the invention, the dose (0.5-1 ml in volume) of 10⁷ TCID₅₀ (tissue culture infectious dose 50) of virus was inoculated via nasal cavity into Rhesus monkeys and Cynomogus Macaques free-living in China.

In the other practical scheme of the invention, the dose of 10⁶ TCID₅₀ (0.5-1 ml in volume) of virus was inoculated via the bronchi into Rhesus monkeys and Cynomogus Macaques free-living in China.

In another practical scheme of the invention, the dose of 10⁵ TCID₅₀ (0.5-1 ml in volume) of virus was inoculated intravenously into Rhesus monkeys and Cynomogus Macaques free-living in China. Actually, the three infectious routes could be selected arbitrarily.

Infected animals with route above in the invention showed fever on the 3th-5th day after inoculation of SARS-CoV. the SARS-CoV RNA was detected positive from 5^{th} day after inoculation of SARS-CoV by using nested RT-PCR technique in pharyngeal swab samples, and lasted for 16 days. Virus isolation was positive during the period.

The infections in humans are mainly through human-to-human contact and aerosol mode, and patients showed high fever and lung infection. The experimental techniques for infection were firstly used by inoculation of respiratory duct with cultured virus, including via nasal cavity and bronchi injection, and also to be infected by mainline. Both Rhesus monkeys and Cynomogus Macaques were successfully infected with a low dose of virus. The Marmoset (Callithrix jacchus) was not infected successfully using the same methods. The results revealed that the sensitivities of non-human primate to SARS-CoV were different.

Another aspect of this invention is related to the SARS primate animal model that are used for studies on the process of viral infection in vivo, viral pathogenesis and body responses.

At present, a lot of problems on SARS remain unknown, including the targeting cells, tissues and organs, the affection on immune system and the responses from immune cells, etc. Any samples that could not be collected from human body can be carried on selecting samples from any index of time-dynamics by using the model, showed trail, and use for relevant condition co-pathogen any causative agent can be studied with SARS as in the model.

One more aspect of this invention is related to the SARS primate animal model that is applicable in screening medicines and evaluating vaccines against SARS-CoV.

The virological and serological data could be detected from both Rhesus monkeys and cynomogus macaques after inoculation of SARS-CoV, for instance, the SARS coronavirus-specific IgM and IgG were begun to detect positive in the serum of monkeys in certain period, viruses could be isolated, and the SARS-CoV RNA was detected positive for 10-15 days using nested RT-PCR in pharyngeal swab samples. The virological and serological criteria would be served uniquely for screening for medicines and vaccines against SARS-CoV, namely, the model is able to evaluate the effectiveness of any anti-SARS-CoV medicines, including traditional Chinese herbs. The effect and side-effect could be displayed by the changes of related data in animal model.

There are no other laboratory animals close genetically to humans more than non-human primates, therefore, this model could play a unique role in evaluating vaccines against SARS-CoV.

It is to be understood that both the foregoing general description and the following detailed description are exemplary and explanatory and are intended to provide further explanation of the invention as claimed.

### BRIEF DESCRIPTION OF THE DRAWINGS

The accompanying drawings are included to provide a further understanding of the invention. The drawings illustrate embodiments of the invention and together with the description serve to explain the principles of the embodiments of the invention.

FIG. 1 shows the nested RT-PCR products of SARS-CoV from samples of Rhesus monkeys. From left to right, lane 1: the throat swab of #883 on day 5 after inoculation with SARS-CoV. Lane 2: the throat swab of #900 on day 5 after inoculation with SARS-CoV. Lane 3: DL2000 marker (2000, 1000, 750, 500, 250 and 100 bp). Lane 4: Negative control. Lane 5: Positive control.

FIG. 2A shows a pathologic tissue section of the lung tissue from #883 monkey that was one from animal model in the invention.

FIG. 2B shows a tissue section of the normal lung tissue from a healthy monkey.

### DETAILED DESCRIPTION

Advantages of the present invention will become more apparent from the detailed description given herein after. However, it should be understood that the detailed description and specific examples, while indicating preferred embodiments of the invention, are given by way of illustration only, since various changes and modifications within the spirit and scope of the invention will become apparent to those skilled in the art from this detailed description.

### The First Example of Exploitation of the Stock of SARS-CoV

The SARS virus was isolated from a typical SARS patient in China and cultured with Vero-E6 cells. The virus was identified and characterized by RT-PCR and electron microscopy. The stock of the SARS-CoV isolated in China has deposited in CGMCC on 26 Jun., 2003, a international institute for storage that was recognized internationally in the process of patent for microbiologic storage according to Budapest Treaty, and the registration No. 0962 was given. The international institute for storage is located: 13 Beiyitiao Zhongguanchun, District of Haidian, Beijing, P.R.China. The TCID₅₀ of SARS-CoV is 10⁶ pfu/ml

### The Second Example of Exploitation The Establishment of Animal Model

1. Virus Inoculation

Two monkeys, 13-months old, were obtained from the Institute of Medical Biology, Chinese Academy of Medical Sciences & School of Basic Medicine (Kunming, China) The female was numbered as #900 and the male was #883. Before inoculation of SARS virus, the monkeys were examined according to the national microbiological and parasite SPF (specific pathogen-free) standard and were verified free of anti-SARS antibody. 10⁷ TCID50 of virus described in Applied item 1 was inoculated into monkey #900 (1 ml in volume) and #883 (0.5 ml in volume). Both monkeys were housed in biosafety 3 (P3+) animal laboratory that was specifically designed for SARS research. The animal experiments were carried out strictly following the Chinese National Standard Protocols of Animal Manipulations.

2. Observations on Clinical Signs of Monkeys After Inoculation of SARS-CoV

The animals were subjected to daily measurement of anal temperature, the 2^{nd} to 3^{rd} day after inoculation of the SARS virus, both macaques showed a temporary rise in temperature, which initially peaked to 39° C., but subsequently subsided to 38° C. We have never observed any abnormal changes in leukocyte count, nor did we observe any clinical symptoms or signs, such as cough, upper respiratory tract catarrh, vomiting, rash, diarrhea, shortness of breath, dyspnea, or notable loss of appetite.

2. Virological Monitoring for Monkeys After Inoculation of SARS-CoV

For isolation of SARS-CoV, on day 2, 5 and 7 after inoculation of the virus, the pharyngeal swab and nasal swab samples from the infected macaques were inoculated onto Vero E6 Cells, and cultured in DMEM (Gibco, USA). At least, observation on cytopathological changes (CPE) should keep three to fourth passages of the SARS-CoV on Vero E6 cells, [the detailed method refers to the technique in cell culture in <The virological and experimental techniques in medicine>, Huang Zhenxiang, et al. Beijing, Science publish Co. p 130-141, 1990]. Both cells showed CPE and no CPE appeared were identified by IFA technique. Viruses were isolated from both #900 and #883.

3. Detection of RNAs of SARS-CoV in Animal Model by Nested RT-PCR

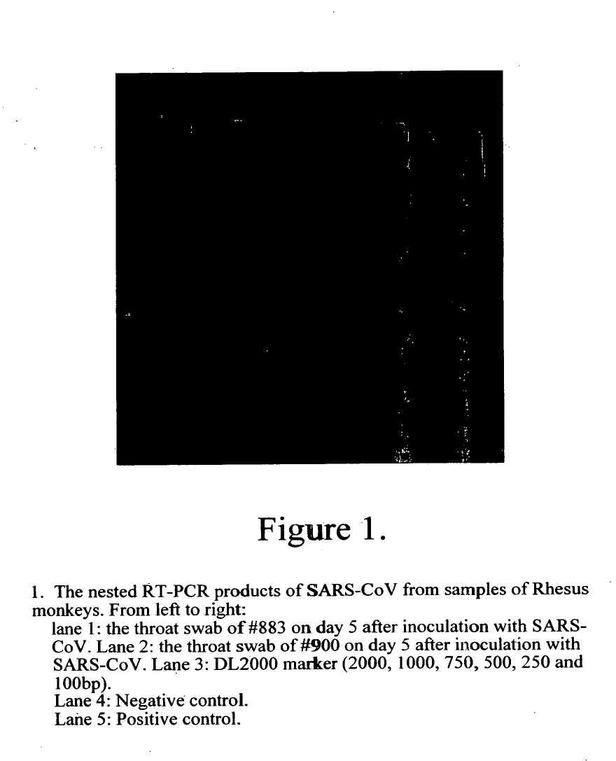

To test the existence and replication of SARS-CoV in monkeys, the pharyngeal swab and nasal swab samples were collected from infected monkeys on the first day after inoculation and were tested for SARS-CoV by nested RT-PCR(QIAamp Viral RNA Mini Kit, Qiagen, Germany), using of the outer primer pair, (5′-GCTGCATTGGTTTGTTATATCGTTATGC-3′) and (5′-ATACAGAATACATAGATTGCTGTTATCC-3′) and the inner primer pair, (5′-TCACTTGCTTCCGTTGAGGTAGCCAGCGTGGTGGTTCATACAA-3′), and (5′-GGTCGGATGTTACAGCGTCTCCCGGCAGAAAGCTGTAAGCT-3′. The products from second-round PCR were run in 2% agarose gel, the result displayed in FIG. 1. SARS coronavirus RNAs were detected in the pharyngeal swab and nasal swab samples of infected macaques on the fifth day of inoculation by nested RT-PCR. A 797 bp fragment of the SARS-CoV was amplified from the pharyngeal and nasal samples. Positive detection lasted as long as 16 days-post inoculation (d.p.i.). The positive control was SARS-CoV cultured in Vero cells and negative control was from normal monkey. The result demonstrated that the monkeys were infected with SARS-CoV.

The results above revealed that the Rhesus monkeys were infected effectively with SARS-CoV via nasal cavity, and there were replication and release of viruses in animal model in the invention.

4. Pathological Examination on Animal Model

On the 7^{th} day after inoculation with SARS-CoV, lung tissues were taken by BADD tube from the infected monkeys. For preparation of paraffin blocks, tissues were fixed using 10% neutral buffered formalin fixation, and processed in the routine procedures. H-E stains were used for identifying changes of the lungs.

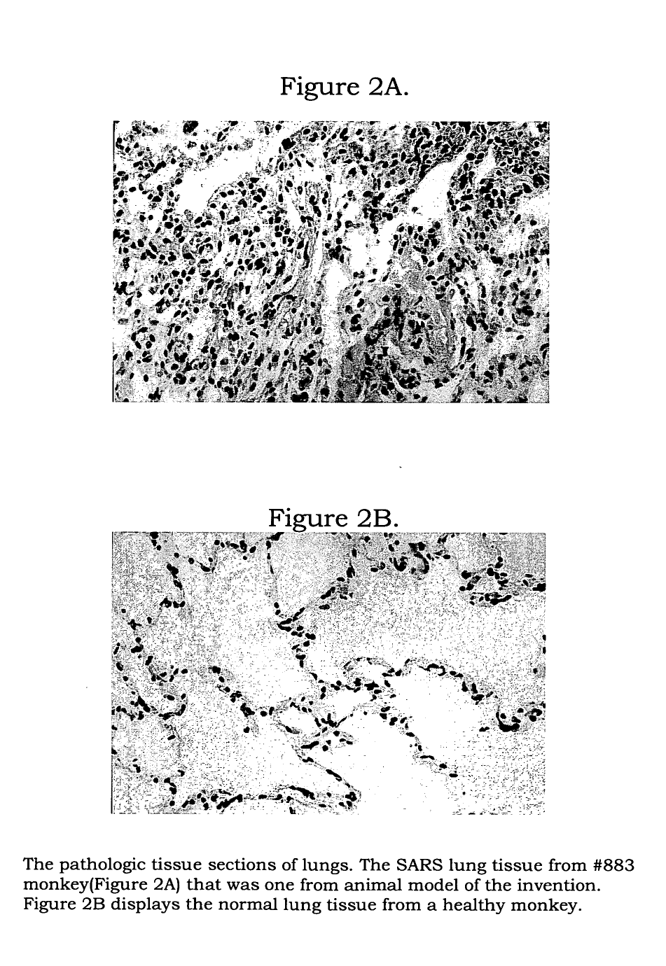

As a result of observation, lungs from both monkeys demonstrated acute hemorrhagic interstitial pneumonia. There was marked infiltration of lymphocytes and macrophages in the nodular area of lungs accompanied by fragmentation of reticulin and elastic fibers of the alveolar wall, Massive or tiny hemorrhage was frequently observed (FIGS. 2A; 2B showed normal control lung tissue). The pathological result demonstrated that the animal model in the invention was able to simulate efficiently SARS-CoV in infection process of primates.

5. Serologic Detection on SARS-CoV Related Antibody in Animal Model

On 5^{th}, 9^{th}, 13^{th}, 17^{th}, 20^{th} day after virus inoculation, the Rhesus monkeys were anaesthetized with ketamine and 1 mL of blood sample was collected from the inguinal veins from each. SARS-CoV specific antibodies in sera were tested by ELISA to observe the immune response to SARS-CoV. (IgG and IgM ELISA kit: Huada-Gibiai Ltd, Beijing).

The results revealed that the serum samples obtained from both animals prior to inoculation were negative for anti-SARS antibodies. IgM from both monkeys were positive on 5^{th} day; IgG were positive in 2 infected-monkeys on the 17^{th}, 20^{th}, 24^{th}, 28^{th}, 32^{nd} day after viral inoculation.

### Experimental Results

The inventors for the first time have successfully established the SARS animal model using Rhesus monkeys and Cynomogus Macaques free-living in China, that were inoculated with SARS-CoV isolated from patient in China. Although all animals show light evident clinical signs except for a transient fever 2 to 3 days after inoculation of SARS SARS-CoV, the count of WBC in monkeys were not high, this was similar to SARS patients. The SARS virus RNA was detected positive using nested RT-PCR in pharyngeal swab samples 5 days following inoculation, the SARS coronavirus-specific IgG began to be detected positive in the serum of monkeys 11-15 days after inoculation, and viral isolation showed replication of SARS-CoV. In addition, pathologic findings of lung tissues showed broadened alveolar septa, epithelial hyperplasia, capillary vessel dilatation and congestion, and interstitial lymphocyte infiltration on the seventh day after inoculation. Taken together, the pathological changes, immune response and virus excretion in the SARS animal model may provide insight into the mechanism of SARS infection and may greatly facilitate the screening of anti-SARS drug and vaccine evaluation.

It is to be understood that the foregoing descriptions and specific embodiments shown herein are merely illustrative of the best mode of the invention and the principles thereof, and that modifications and additions may be easily made by those skilled in the art without departing for the spirit and scope of the invention, which is therefore understood to be limited only by the scope of the appended claims.

1. A primate animal model for SARS-CoV.

2. The primate animal model for SARS-CoV according to claim 1, wherein said primate animal model for SARS-CoV is the establishment of SARS model by using Rhesus monkeys free-living in China.

3. The primate animal model for SARS-CoV according to claim 1, wherein said primate animal model for SARS-CoV is the establishment of SARS model by using Cynomogus Macaques free-living in China.

4. The primate animal model for SARS-CoV according to claim 2, wherein the Rhesus monkeys or Cynomogus Macaques free-living in China are infected by at least through one route for inoculation:
1) Inoculated via nasal cavity, the dose is 0.5-1 ml 10⁷ TCID₅₀;
2) Inoculated via the bronchi, the dose is 0.5-1 ml 10⁶ TCID₅₀; or
3) Inoculated intravenously, the dose is 0.5-1 ml 10⁵ TCID₅₀;
and the infected animals showed fever on days 2-3 after inoculation of SARS-CoV, the SARS-CoV RNA was detected positive from a 5^{th} day after inoculation of SARS-CoV by using nested RT-PCR technique in pharyngeal swab samples, and lasted for 16 days, and virus isolation was positive during the period.

5. The strain of SARS-CoV that was used in establishment of SARS primate model for claim 1, wherein the registration No. is CGMCCC0962.

6. A method used in establishment of the SARS primate model of claim 1, including at least one step by routes as follows:
4) inoculation via nasal cavity, the dose being 0.5-1 ml 10⁷ TCID₅₀;
5) inoculation via the bronchi, the dose being 0.5-1 ml 10⁶ TCID₅₀;
6) Inoculated intravenously, the dose being 0.5-1 ml 10⁵ TCID₅₀;
and the animals showed fever on days 2-3 after inoculation of SARS-CoV, the SARS-CoV RNA was detected positive from a 5^{th} day after inoculation of SARS-CoV by using nested RT-PCR technique in pharyngeal swab samples, and lasted for 16 days, and virus isolation was positive during the period.

7. The method according to claim 6, wherein the said method also includes as follows:
7) a step of virologic detection in SARS animal model;
8) a step of serologic detection in SARS animal model;
9) a step of detection for neutralizing antibody in SARS animal model; and/or
10) a step of histo-pathologic examination in SARS animal model.

8. The method according to claim 7, wherein said serologic antibody detection is IFA and/or ELISA tests.

9. An application of the SARS primate animal model according to claim 1, wherein the animal model is applicable for screening medicines against SARS-CoV.

10. An application of the SARS primate animal model according to claim 1, wherein the animal model is applicable for evaluating vaccines against SARS-CoV.
